(19) United States
(12) Patent Application Publication
(10) Pub. No.: US 2005/0186633 A9
(43) Pub. Date: Aug. 25, 2005

(54) In vitro gastrointestinal model system and uses thereof

(76) Inventors: Christophe Lacroix, Kilchberg (CH); Gwenaelle LeBlay, Zurich (CH); Cecile Cinquin, Quebec (CA); Ismail Fliss, Ste-Foy (CA)

Correspondence Address: DOWELL & DOWELL, P.C.;Suite 309, 1215 Jefferson Davis Highway, Arlington, VA, 22202-3124, US

(21) Appl. No.: 10/436,976

(22) Filed: May 14, 2003

Related U.S. Application Data
(60) Provisional application No. 60/380,290, filed on May 15, 2002.

(30) Foreign Application Priority Data
May 15, 2002 (CA) 2,386,554

(51) Int. Cl. G01N 33/53

(52) U.S. Cl. 435/7.1

(57) ABSTRACT

A system utilizing cell immobilization in anaerobic continuous-flow cultures for modelling the gastrointestinal system is described. Microbial cells derived from flora, e.g. in fresh faecal samples, are used as the source of inocula for immobilisation in a mixed gel of gellan and xanthan. The beads produced are then introduced in a single or multi-stage chemostat fed with a nutrient media, and the composition and metabolic activities of the flora are monitored over time in reactors operated with conditions simulating the characteristics of different segments of the gastrointestinal tract. The conditions of this intestinal fermentation model are more akin to that for the gastrointestinal system, in which cells are naturally in the immobilized state, entrapped in fibrous particles or forming biofilms on the intestine epithelium. A use of such a system for studying various aspects of the gastrointestinal tract is also described.

### CROSS-REFERENCE TO RELATED APPLICATIONS

This application claims the benefit of U.S. Provisional Patent Application Serial No. 60/380,290, filed May 15, 2002, which is incorporated by reference herein in its entirety.

### FIELD OF THE INVENTION

The invention relates to an in vitro culture system and more particularly relates to an in vitro culture system to model the gastrointestinal tract, and uses thereof.

### BACKGROUND OF THE INVENTION

One of the most promising areas for the development of functional foods lies in the modification of the activity of the gastrointestinal tract by use of probiotics, prebiotics and synbiotics (Salminen et al., 1998). As such, the continued study of the health benefits of functional foods in both diseased and healthy populations is an important and necessary area of research and development. To understand the potential value of these functional foods and to be able to develop new approaches, it is necessary to study the human intestinal flora. Different in vitro approaches have been used to measure the efficacy of probiotics and prebiotics in adult colonic flora including both batch and continuous culture systems (Wang & Gibson, 1993; Gibson & Wang, 1994). These latter (chemostats) systems can be used to simulate the intestinal conditions more closely than batch culture systems (Veilleux & Rowland, 1981; Freter et al., 1983). By varying dilution rates and other parameters, conditions for growth can be determined under steady-state conditions. Multistage chemostats have also been used as efficient “gut models” in that each vessel represents a different physicochemical region of the intestine (Gibson & Fuller, 2000; Macfarlane et al., 1998, Gibson et al., 1988). However, such systems are lacking in certain aspects and as a result do not reflect the conditions of the gastrointestinal tract with sufficient accuracy and as a result cannot fully contribute to its study. As such, there is a need for an improved in vitro system to model the gastrointestinal tract.

### SUMMARY OF THE INVENTION

The invention relates to an improved in vitro system to model the gastrointestinal system.

Accordingly, in a first aspect, the invention provides an in vitro gastrointestinal model system comprising immobilized microbial cells, such as bacterial cells.

In an embodiment, the microbial cells are derived from faecal flora.

In an embodiment, the microbial cells are immobilized on a matrix comprising a gel. In an embodiment, the matrix comprises gel beads. In an embodiment, the gel is a mixed gel comprising a first gel and a second gel. In an embodiment, the first gel is gellan. In an embodiment, the second gel is xanthan. In an embodiment, the first and second gels are present in a ratio of about 10:1 first gel:second gel. In an embodiment, the gel is obtained from a solution of about 2.5% w/v gellan and about 0.25% xanthan. In an embodiment, the solution further comprises about 0.2% sodium citrate.

In an embodiment, the above noted system has a high cell density. In an embodiment, the cell density is greater than about 10⁹ CFU/ml. In a further embodiment, the cell density is about 10¹⁰ CFU/ml or greater. In a further embodiment, the cell density is about 10¹¹ CFU/ml or greater.

In an embodiment, the microbial cells comprise an anaerobe and a facultative anaerobe. In an embodiment, the anaerobe is selected from the group consisting of Bacteroides fragilis, Bifidobacterium sp., and Clostridium sp.. In an embodiment, the facultative anaerobe is selected from the group consisting of Enterobacteriaceae, Streptococcus sp., Lactobacillus sp., and Staphylococcus sp.

In an embodiment, the above noted system comprises a culture condition having an average pH selected from the group consisting of about 5.7, about 6.2, and about 6.8.

In an embodiment, the above noted system has an increased level of at least one characteristic relative to a corresponding free-cell system, wherein said characteristic is selected from the group consisting of:
(a) cell density;
(b) cell stability;
(c) cell reactivity with components in said system;
(d) cell protection from shear stress;
(e) cell protection from oxygen stress;
(f) resistance to bacterial contamination;
(g) resistance to phage contamination;
(h) resistance of cells to frozen storage; and
(i) any combination of (a) to (h).

In an embodiment, the stability is based on prolonged cell viability and/or prolonged retention of a plasmid-encoded phenotype.

In another aspect, the invention further provides a method of determining the effect of an element on the gastrointestinal tract or on gastrointestinal flora, said method comprising: (a) introducing said element into the above-noted system; and (b) determining whether any change occurs in any characteristic or feature/function of interest of said system in the presence of said element or subsequent to the introduction of said element into said system, wherein said change is indicative that said element has an effect on the gastrointestinal tract or on gastrointestinal flora.

In another aspect, the invention further provides a use of the above-noted system for the study of the effect of an element on the gastrointestinal tract and/or on gastrointestinal flora. In an embodiment, said element is selected from the group consisting of:
(a) bacteria;
(b) a substrate;
(c) a chemical substance; and
(d) any combination of (a) to (c).

In an embodiment, the bacteria are selected from the group consisting of probiotics and pathogens. In an embodiment, the substrate is selected from the group consisting of foodstuffs, prebiotics, synbiotics and dietary fibers. In an embodiment, the chemical substance is selected from the group consisting of drugs (e.g. antibiotics), lactoferrin, and bacterioricins.

The invention further provides a method to use the above noted system to study the effects of the above-noted elements on the on the on the gastrointestinal tract and/or on gastrointestinal flora. In an embodiment such a method comprises culturing the immobilized cells in the above-noted system and controlling or adjusting culture conditions with regard to the element or functional aspect and effect thereof on the gastrointestinal tract and/or the gastrointestinal flora, which is being studied.

### BRIEF DESCRIPTION OF THE DRAWINGS

FIG. 1: Comparison of bacterial populations used for immobilization (faeces) and measured in gel beads at pseudo-steady states during continuous culture with changing conditions (PCS: proximal colon simulation; TCS: transverse colon simulation; DCS: distal colon simulation).

FIG. 2: Comparison of bacterial populations used for immobilization (faeces) and measured in effluent media at pseudo-steady states during continuous culture with changing conditions (PCS: proximal colon simulation; TCS: transverse colon simulation; DCS: distal colon simulation).

FIG. 3: Molar proportions of individual short chain fatty acids in the fermentation effluent at pseudo-steady state during continuous culture with changing conditions (PCS: proximal colon simulation; TCS: transverse colon simulation; DCS: distal colon).

### DETAILED DESCRIPTION OF THE INVENTION

Presently, the only in vitro models of the gastrointestinal flora use free-cell fermentations, both with batch and continuous cultures. “Free-cell” systems differ from those of the invention in that the systems of the invention comprise cells which have been immobilized to/with a matrix. However, when steady-state is reached, the total bacterial number in liquid chemostats (10⁹ CFU/ml) does not reach the high concentrations of bacterial populations (10¹⁰-10¹¹ CFU/g wet weight) observed in faeces and colonic contents (Rumney & Rowland, 1992). Described herein is the use of immobilized cell technology for a gastrointestinal model system, which provides an environment more akin to that of the gastrointestinal tract compared to conventional liquid cultures. Certain reports have studied selection and optimization of biopolymer gel matrices possessing a high mechanical stability during long-term continuous lactic fermentation (Artignan et al., 1997; Lamboley et al., 1999). Compared to these classical models, the immobilized cell technology developed in this study for modelling gastrointestinal fermentation has, for example, the following characteristics and advantages:
high cell density in gel beads and in the reactor effluent (greater than about 10⁹ CFU/ml or g and up to about 10¹⁰ CFU/ml or g) which is more representative of the high cell concentrations in human colon and faeces;
high stability of the system over long periods of experimentation which allows for testing different conditions during the same continuous culture experiment with the same beads and faecal flora;
high stability and reactivity of the intestinal fermentation;
good protection of sensitive bacteria from shear and oxygen stresses;
high resistance to bacterial and phage contamination;
stable storage of immobilized faecal flora in a frozen state and utilisation of the pre-colonised beads containing the same flora for several experiments; and
provides an intestinal in vitro fermentation model more akin to that of the gastrointestinal system.

Advantages are demonstrated herein using this technology, including high productivity, improved control of microbial populations, high stability of the continuous process over extended periods experimented (up to 90 days), stabilization of plasmid encoded traits of the strains, resistance to bacterial and phage contamination. These high performances compared with classical free-cell fermentations are partly explained by the very high cell density retained in the reactor, typically ranging from 2×10¹⁰ to 2×10¹¹ CFU/mL and the discrete localization of immobilized cells. In an embodiment, the system of the invention has a cell density of greater than that of a corresponding free-cell system. In an embodiment, system of the invention has a cell density greater than about 10⁹ CFU/ml. In further embodiments, the cell density is greater than about 2×10⁹, 4×10⁹, 6×10⁹, 8×10⁹, 10¹⁰, 2×10¹⁰, 4×10¹⁰, 6×10¹⁰, 8×10¹⁰, or 10¹¹ CFU/ml. Cell immobilization and the formation of an active peripheral cell layer in gel beads with very high cell density, particularly in microcolonies where cells are very closely packed, may also result in improved cell to cell communication and increased expression of cell-density dependent genes (quorum sensing).

The invention thus provides a system utilizing immobilized cell technology, which may be used, for example, for the control and modulation of physiology and especially probiotic characteristics of lactic acid bacteria, bifidobacteria and other probiotic cultures.

In an aspect, the invention entails the use of cell immobilization in anaerobic continuous-flow cultures for modelling gastrointestinal flora. “Anearobic” as used herein refers to culturing the cells under conditions which are substantially free of oxygen. Fresh faecal samples may be used as the source of inocula for immobilization of a suitable matrix, e.g. a gel. In an embodiment, the gel is a mixed gel, comprising a first gel and a second gel. In a further embodiment, the first gel is gellan. In a further embodiment, the second gel is xanthan. The beads produced are then introduced in a suitable culture system, e.g. a single or multi-stage chemostat fed with a nutrient media. The composition and metabolic activities of the flora may be monitored at intervals over a period of time, for example daily during several weeks, in reactors operated with conditions simulating the characteristics of different segments of the gastrointestinal tract. The conditions of this intestinal fermentation model are more akin to that of the gastrointestinal system, in which cells are naturally in the immobilized state, entrapped in fibrous particles or forming biofilms on the intestine epithelium.

The system of the invention may for example be used to study the composition and activity of the gastrointestinal flora under different environmental conditions, or to test the effects of a variety of components such as:
bacteria (e.g. probiotics, pathogens etc.)
substrates (e.g. prebiotics, dietary fibers etc.)
chemical substances (e.g. antibiotics, lactoferrin, bacteriocins etc.)

It may also be used in a gastrointestinal model system (such as Digestar™) in the colonic segment to simulate more closely intestinal fermentation.

An in vitro model system of gastrointestinal flora of the invention may be used to study the effects of different factors on both composition and metabolic activities of the flora. In vitro models are used to study the mixed bacterial populations of the large intestine. They provide a reproducible baseline for studying the ecology of the gut ecosystem, particularly the changes induced after perturbation of the flora by diet, drugs and a large variety of products and chemicals. The in vitro model of the invention may be used for developing and testing probiotic, prebiotic or synbiotic foods and their effects on the gastrointestinal microflora. It may also be used to test intestinal flora sampled from an ill animal (e.g. a mammal [e.g. a human]) or unbalanced flora and to assess the effect of different treatments that could be used to balance the gastrointestinal flora and eventually treat the patient.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term., substantially equivalent to the phrase “including, but not limited to”. The following examples are illustrative of various aspects of the invention, and do not limit the broad aspects of the invention as disclosed herein.

### EXAMPLES

### Example 1

### Culture System Setup

The continuous system comprises a mechanically-stirred fermentor connected to a stirred feedstock vessel containing sterile feed medium at 4° C., and to an effluent vessel for collecting the effluent. Automatic timers control peristaltic pumps that pump nutrient medium from feedstock vessel into the culture vessel and culture content out into the effluent vessel. The fermentor is maintained, at 37° C. under CO₂ oxygen-free atmosphere, and the pH is automatically controlled by addition of base.

### Example 2

### Immobilization of Faecal Flora

Fresh faecal samples are used as the source of inocula for immobilization and continuous culture. A special attention (anaerobiosis) is paid on preserving the viability and integrity of the faecal flora during sampling.

Faecal microflora are immobilized in 1-2 mm diameter gellan/xanthan mixed gel beads, using the double phase dispersion process previously described previously (Lamboley et al., 1997; Audet et al., 1989; Camelin et al., 1993). The mixed gel produced by dissolving gellan gum (2.5% w/v) and xanthan gum (0.25% w/v) in sodium citrate solution (0.2%) is a good entrapment matrix for temperature-sensitive cells, with good mechanical properties required for long term stability during continuous culture with immobilized cells. It is stabilized by a large variety of monovalent and divalent cations, which are present in culture broths. Gellan also exhibits a useful synergism with other polymers, such as xanthan. Applicants used a mixed gel of gellan and xanthan to increase strength and decrease brittleness of the gel, which are two important characteristics for bead stability in bioreactors.

### Example 3

### Culture of Immobilized Cells and Properties Observed in the System

The fermentor was inoculated with 30% (v/v) of beads (range from 0.5 to 50% v/v) which were precolonized during four successive pH-control batch cultures (4×12 h). The continuous culture was then conducted for 54 days. The pH was successively controlled at 5.7; 6.2 and 6.8 with retention times set at 4, 8 and 12 hours, respectively, in order to simulate the proximal (PCS), transverse (TCS) and distal colons (DCS). The metabolic activity was analysed and the free cell populations were enumerated on each day, whereas the immobilized populations were enumerated once a week.

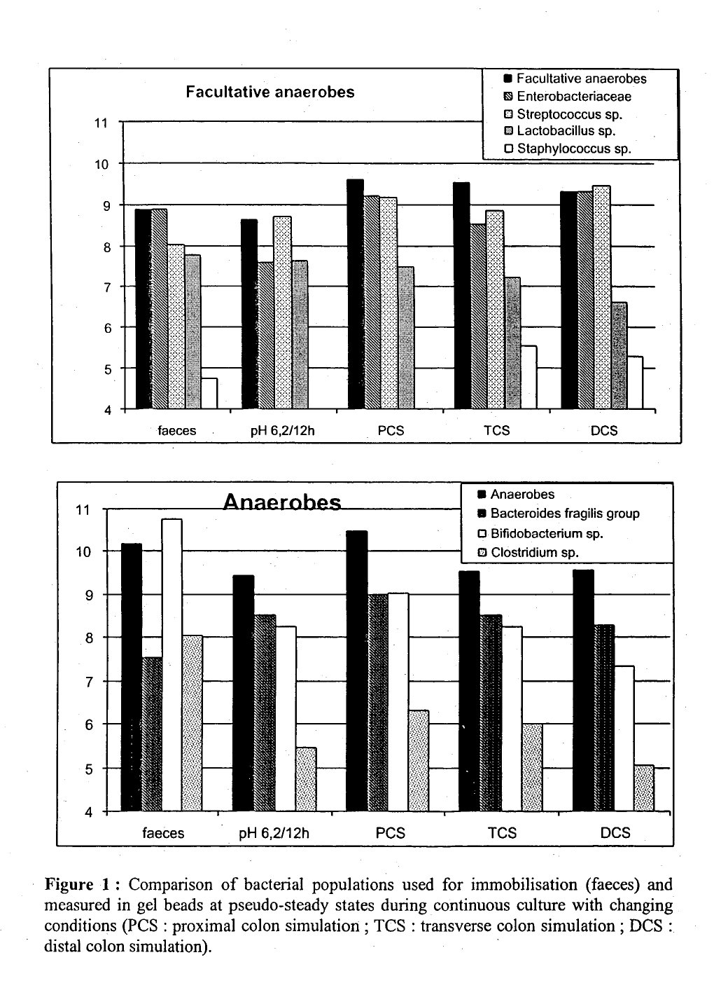

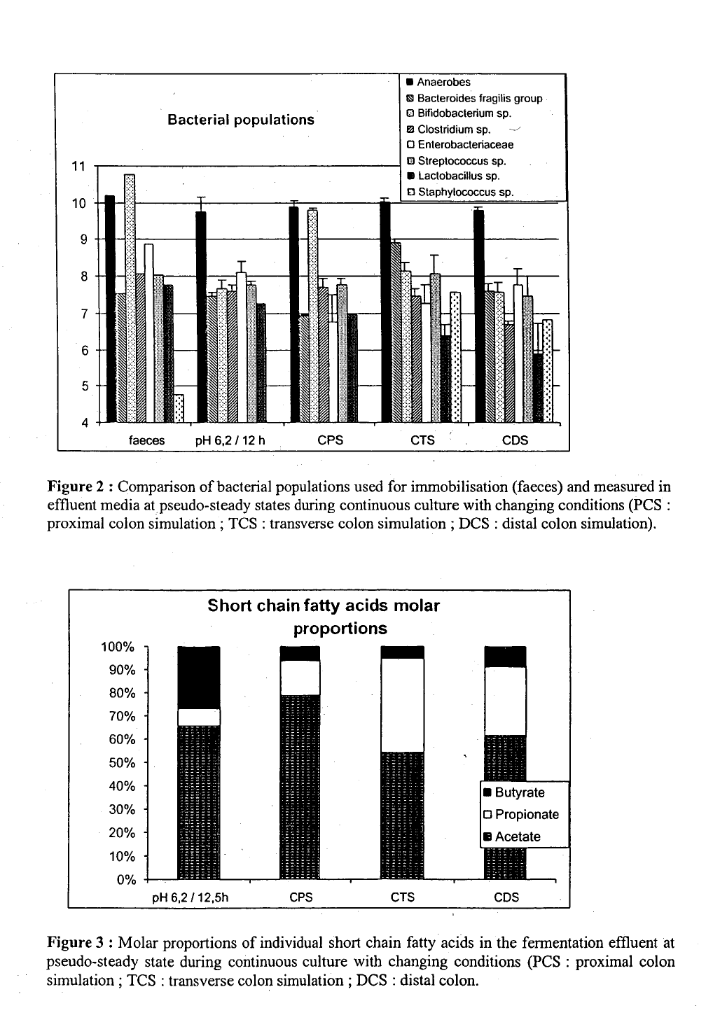

High cell survival for the major bacterial groups present in baby's faeces was maintained during the immobilization process. For a total anaerobic count of 10.75 Log CFU/g measured in faeces before immobilization, 9.99 Log CFU/g were recovered after immobilization in the gel beads. After four weeks culture, gel beads were highly colonised with all the bacterial populations studied and their relative proportions were maintained during the whole fermentation period and little affected by changing pH and residence time conditions (FIG. 1). In the effluent, anaerobes outnumber facultative anaerobes by a factor of 100 as usually described in the colonic contents. Contrary to the immobilized populations, free-cell populations were strongly affected by the fermentation parameters (FIG. 2) Compared to the stabilization period (pH 6.2, retention time of 12 h), the PCS was characterized by high Bifidobacterium sp. concentrations that exceeded the other populations by 2 Log. They then gradually decreased from the PCS to the DCS as did the Lactobacillus sp. and Clostridium sp. populations. The Bacteroides fragilis group increased in the CPS where they became the predominant population but decreased in the CTS. Enterobacteriaceae and Staphylococcus sp. increased from the PCS to the DCS where Enterobacteriaceae cell counts reached the concentrations of Bifidobacterium sp. As for the bacterial populations, the metabolite production was affected by tested fermentation conditions. In particular the short chain fatty acid concentration ratios were greatly altered (FIG. 3). The stabilization period was characterized by a high proportion of butyrate compared to the other tested conditions. The PCS condition was characterized by a high proportion of acetate whereas the TCS induced a high proportion of propionate, and the data obtained in the DC were intermediate.

The colonic fermentation system obtained was very stable during long term fermentation. A good correlation was observed between the bacterial concentrations obtained in this system and the data obtained from infant faecal flora. (Kleessen et al., 1995; Grönlund et al., 1999) and infant-flora associated mice (Hentges et al., 1992).

A high viability of the different bacterial populations during frozen storage (−80° C.) of the pre-colonised beads in a cryoprotective solution was also observed during more than 3 months storage. The storage of the pre-colonised beads allows the utilisation of the same flora in several experiments.

All references cited above or in the References section below are herein incorporated by reference.

### References

Audet P, Lacroix C. Two phase dispersion process for the production of biopolymer gel beads. Process Biochem.1989; 24 : 217-226.
Artignan, Corrieu, Lacroix. Rheological study of pure and mixed K-carrageenan gels in lactic acid fermentation conditions.J. Text. Studies 1997; 28: 47. 9 je n'ai pas trouve cette ref
Camelin I, Lacroix C, Paquin C, Prevost H, Cachon R, Divies C. Effect of chelatants on gellan gel rheological properties and setting temperature for immobilization of living bifidobacteria. Biotechnol. Prog. 1993; 9: 291-7.
Freter R, Stauffer E, Cleven D, Holdeman L V, Moore W E. Continuous-flow cultures as in vitro models of the ecology of large intestinal flora. Infect. Immun. 1983; 39: 666-75.
Gibson G R, Cummings J H, Macfarlane G T. Use of a three-stage continuous culture system to study the effect of mucin on dissimilatory sulfate reduction and methanogenesis by mixed populations of human gut bacteria. Appl. Environ. Microbiol. 1988; 54: 2750-5.
Gibson G R, Fuller R. Aspects of in vitro and in vivo research approaches directed toward identifying probiotics and prebiotics for human use. J. Nutr. 2000;130(2S Suppl): 391S-395S.
Gibson G R, Wang X. Enrichment of bifidobacteria from human gut contents by oligofructose using continuous culture. FEMS Microbiol. Lett. 1994; 118:121-7.
Gronlund M M, Lehtonen O P, Eerola E, Kero P. Fecal microflora in healthy infants born by different methods of delivery: permanent changes in intestinal flora after cesarean delivery. J. Pediatr. Gastroenterol. Nutr. 1999; 28: 19-25.
Hentges D J, Marsh W W, Petschow B W, Thal W R, Carter M K. Influence of infant diets on the ecology of the intestinal tract of human flora-associated mice. J. Pediatr. Gastroenterol. Nutr. 1992; 14:146-52.
Kleessen B, Bunke H, Tovar K, Noack J, Sawatzki G. Influence of two infant formulas and human milk on the development of the faecal flora in newborn infants. Acta Paediatr. 1995;84: 1347-56.
Lamboley L., Lacroix C., Champagne C. P., Vuillemard J. C. Continuous mixed strain mesophilic lactic stater production in supplemented whey permeate medium using immobilized cell technology. Biotechnol. Bioeng., 1997; 56: 502-516.
Lamboley L, Lacroix C, Artignan J M, Champagne C P, Vuillemard J C. Long-term mechanical and biological stability of an immobilized cell reactor for continuous mixed-strain mesophilic lactic starter production in whey permeate Biotechnol. Prog. 1999;15: 646-54.
Macfarlane G T, Macfarlane S, Gibson G R. Validation of a three-Stage compound continuous culture system for investigating the effect of retention time on the ecology and metabolism of bacteria in the human colon Microb. Ecol. 1998; 35 :180-7.
Rumney C J, Rowland I R. In vivo and in vitro models of the human colonic flora. Crit Rev. Food Sci. Nutr. 1992; 31: 299-331.
Salminen S, Bouley C, Boutron-Ruault M C, Cummings J H, Franck A, Gibson G R, Isolauri E, Moreau M C, Roberfroid M, Rowland I. Functional food science and gastrointestinal physiology and function. Br. J. Nutr. 1998; 80 Suppl 1: S147-71.
Veilleux B G, Rowland I. Simulation of the rat intestinal ecosystem using a two-stage continuous culture system. J. Gen. Microbiol. 1981; 123: 103-15.
Wang X, Gibson G R. Effects of the in vitro fermentation of oligofructose and inulin by bacteria growing in the human large intestine. J. Appl. Bacteriol. 1993;75: 373-80.

1. An in vitro gastrointestinal model system comprising immobilized microbial cells.

2. The system of claim 1, wherein said microbial cells comprise bacterial cells.

3. The system of claim 1, wherein said microbial cells are derived from faecal flora.

4. The system of claim 1, wherein said microbial cells are immobilized on a matrix comprising a gel.

5. The system of claim 4, wherein said matrix comprises gel beads.

6. The system of claim 4, wherein said gel is a mixed gel comprising a first gel and a second gel.

7. The system of claim 6, wherein said first gel is gellan.

8. The system of claim 6 wherein said second gel is xanthan.

9. The system of claim 7 wherein said second gel is xanthan.

10. The system of claim 6 wherein said first and second gels are present in a ratio of about 10:1 first gel:second gel.

11. The system of claim 9 wherein said first and second gels are present in a ratio of about 10:1 first gel:second gel.

12. The system of claim 11 wherein said gel is obtained from a solution of about 2.5% w/v gellan and about 0.25% xanthan.

13. The system of claim 12, wherein said solution further comprises about 0.2% sodium citrate.

14. The system of claim 1, having a first cell density which is higher than a second cell density measured in a corresponding free-cell system.

15. The system of claim 14, wherein said first cell density is greater than about 10⁹ CFU/ml.

16. The system of claim 15, wherein said first cell density is about 10¹⁰ CFU/ml or greater.

17. The system of claim 16, wherein said first cell density is about 10¹¹ CFU/ml or greater.

18. The system of claim 1, wherein said microbial cells comprise an anaerobe and a facultative anaerobe.

19. The system of claim 18, wherein said anaerobe is selected from the group consisting of Bacteroides fragilis, Bifidobacterium sp., and Clostridium sp.

20. The system of claim 18, wherein said facultative anaerobe is selected from the group consisting of Entero bacteriaceae, Streptococcus sp., Lactobacillus sp., and Staphylococcus sp.

21. The system of claim 1 wherein said system comprises a culture condition having an average pH selected from the group consisting of about 5.7, about 6.2, and about 6.8.

22. The system of claim 1, wherein said system has an increased level of at least one characteristic relative to a corresponding free-cell system, wherein said characteristic is selected from the group consisting of:
(j) cell density;
(k) cell stability;
(l) cell reactivity with components in said system;
(m) cell protection from shear stress;
(n) cell protection from oxygen stress;
(o) resistance to bacterial contamination;
(p) resistance to phage contamination;
(q) resistance of cells to frozen storage; and
(r) any combination of (a) to (h).

23. The system of claim 22, wherein said stability is based on prolonged cell viability and/or prolonged retention of a plasmid-encoded phenotype.

24. A method of determining the effect of an element on the gastrointestinal tract or on gastrointestinal flora, said method comprising:
(a) introducing said element into the system of claim 1; and
(b) determining whether any change occurs in any characteristic of said system in the presence of said element, wherein said change is indicative that said element has an effect on the gastrointestinal tract or on gastrointestinal flora.

25. The method of claim 24, wherein said element is selected from the group consisting of:
(a) bacteria;
(b) a substrate;
(c) a chemical substance; and
any combination of (a) to (c).

26. The method of claim 25, wherein the bacteria are selected from the group consisting of probiotics and pathogens.

27. The method of claim 25, wherein the substrate is selected from the group consisting of foodstuffs, prebiotics, synbiotics and dietary fibers.

28. The method of claim 25, wherein the chemical substance is selected from the group consisting of drugs, lactoferrin, and bacterioricins.

29. The method of claim 28, wherein the drug is an antibiotic.

30. Use of the system of claim 1 for study of the effect of an element on the gastrointestinal tract and/or on gastrointestinal flora, wherein said element is selected from the group consisting of:
(d) bacteria;
(e) a substrate;
(f) a chemical substance; and
(g) any combination of (a) to (c).

31. The use of claim 24, wherein the bacteria are selected from the group consisting of probiotics and pathogens.

32. The use of claim 23, wherein the substrate is selected from the group consisting of foodstuffs, prebiotics, synbiotics and dietary fibers.

33. The use of claim 23, wherein the chemical substance is selected from the group consisting of drugs, lactoferrin, and bacterioricins.

34. The use of claim 27, wherein the drug is an antibiotic.
